(19) United States
(12) Patent Application Publication
(10) Pub. No.: US 2023/0371877 A1
(43) Pub. Date: Nov. 23, 2023

(54) METHOD FOR ANALYZING MYOCARDIAL ISCHEMIA IN RESTING ELECTROCARDIOGRAM

(71) Applicants: Chun-Lin Wang, Hsinchu County (TW); Chiu-Chi Wei, Hsinchu City (TW); Pin-Hsiang Chang, Hsinchu City (TW)

(72) Inventors: Chun-Lin Wang, Hsinchu County (TW); Chiu-Chi Wei, Hsinchu City (TW); Pin-Hsiang Chang, Hsinchu City (TW); Yu-Jen Lin, Taoyuan City (TW)

(21) Appl. No.: 17/867,473

(22) Filed: Jul. 18, 2022

(30) Foreign Application Priority Data
May 19, 2022 (CN) 202210544562.7
May 19, 2022 (TW) 111118712

(51) Int. Cl. A61B 5/349; G16H 50/30; G16H 50/70

(52) U.S. Cl. CPC A61B 5/349; G16H 50/30; G16H 50/70

(57) ABSTRACT

A method for analyzing myocardial ischemia in resting electrocardiogram comprises establishing the database, grouping in a database, determining the threshold value for the group, processing the to-be-determined resting electrocardiogram, and determining the stenosis degree of the coronary artery.

### BACKGROUND OF THE INVENTION

### 1. Field of the Invention

The present invention relates to a method for analyzing electrocardiogram, particularly to a method for analyzing myocardial ischemia in resting electrocardiogram.

### 2. Description of the Prior Art

Generally, the resting electrocardiogram can detect whether the heart beating rate is too fast or too slow or persistent arrhythmia. Some people who have coronary artery stenosis over 70% do not feel chest pains or tightness when resting unless they are exercising. The resting electrocardiogram is unable to detect paroxysmal coronary artery disease. Thus, the treadmill exercise test is necessary for the stress test regarding the cardiac.

In the normal situation, the heart beating gets faster and the blood pressure gets higher after exercising. If the participant heartbeat gets slower and the blood pressure gets lower after exercising, it indicates that the cardiac may be severely hypoxic. Although the treadmill exercise test is not invasive, it is dangerous for those with potential cardiovascular disease. Once the coronary artery is stenosis, exercise will increase the cardiac load and oxygen consumption, which may easily induce acute myocardial infarction.

Furthermore, some participants are not suitable for the treadmill exercise test due to physical issues such as chest pain from unstable angina pectoris, osteoporosis, being too old to run, or infrequently exercising to the point where running or brisk walking can last just five or six minutes.

We can detect whether having cardiac hypoxia or the cardiovascular obstruction by the treadmill exercise test, but not the stenosis degree of the coronary artery. The stenosis degree of the coronary artery needs to be examined by Computed Tomography (CT).

### SUMMARY OF THE INVENTION

An embodiment of the invention provides a method for analyzing myocardial ischemia in resting electrocardiogram (ECG), comprising: establishing a database, comprising to collect a plurality of resting ECG data, wherein each of the resting ECG data comprises a resting ECG and decomposed into a plurality of Intrinsic Mode Functions (IMFs) as a plurality of corresponding bases C1, C2 . . . , and Cn, wherein |Ci|=√(∫(Ci·Ci)dt), i=1, 2, . . . , n, n is a nature number, and Ctotal=√(Σ|Ci|·|Ci|), wherein the corresponding bases at least comprises C1, C2, and C3; grouping the resting ECG data, wherein a reference group is formed by the resting ECGs without any result of any computer tomography (CT) imaging, wherein a first priority group is formed by the resting ECGs accompanying with a plurality of first results of corresponding CT imaging, and each of the first results comprises a corresponding degree value of a first coronary artery stenosis above an stenosis threshold value, wherein a second priority group is formed by the resting ECGs accompanying with a plurality of second results of corresponding CT imaging, and each of the second results comprises a corresponding degree value of a second coronary artery stenosis below an stenosis threshold value; calculating a corresponding active index of each of the resting ECG data in the database, defined as (W1·|C1|+W2·|C2|+W3·|C3|)/|Ctotal|, wherein W1, W2, W3 are the weighting parameters of C1, C2, C3 respectively, then each active index are summed up to be averaged regarding to the reference group, the first priority group, and the second priority group respectively to obtain a reference grouping threshold value VM1, a first priority grouping threshold value VM2, and a second priority grouping threshold value VM3 respectively, wherein the W1, W2, and W3 are determined according a plurality of rules: (1) (W1+W2+W3)≤1, (2) W1≥W2≥W3, (3) there is a statistically significant difference between VM2 and VM1, and between VM3 and VM1; receiving a to-be-determined resting ECG, and calculating a corresponding to-be-determined active index; and comparing the corresponding to-be-determined active index with the VM1, VM2, and VM3 to determine a stenosis degree of coronary artery.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 illustrates the decomposed bases of a resting ECG;

FIG. 2 is the flow chart of the method for analyzing myocardial ischemia in resting ECG according to the invention;

FIG. 3 illustrates the relative relationships between the grouping threshold values;

FIG. 4 illustrates the relative relationships between the grouping threshold values.

### DETAILED DESCRIPTION OF THE PREFERRED EMBODIMENTS

To more clearly describe a thermal interface material coating method according to the present invention, embodiments of the present invention will be described in detail with reference to the attached drawings hereinafter.

In an embodiment, a computer is employed for data processing. Empirical Mode Decomposition (EMD) is adopted to break down a signal into multiple Intrinsic Mode Functions (IMFs) in Hilbert-Huang Transformation (HHT). And then the IMF is treated as the basis of expansion. There are other alternative methods, such as Short Time Fourier transform, Wavelet Transform, Wigner-Ville distribution, and Choi-Williams distribution.

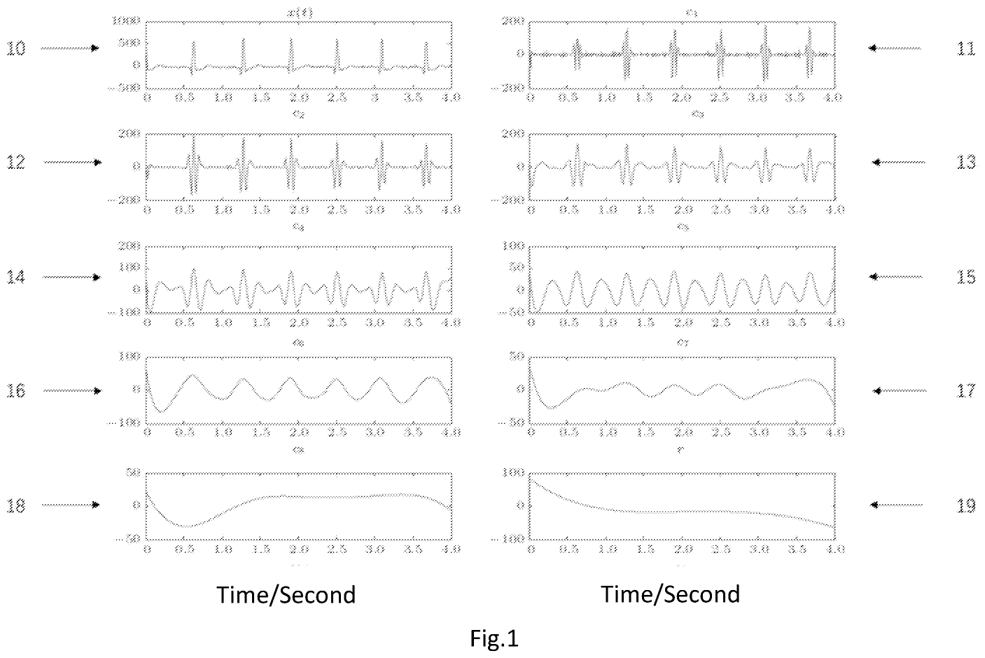

Referred to FIG. 1, an electrocardiography (ECG) monitor is employed to produce a resting ECG. After removing the noises, EMD is adopted to decompose the resting ECG x(t) 10 into the 1st basis (C1) 11, the 2nd basis (C2) 12, the 3rd basis (C3) 13 . . . , the 8^{th} basis (C8), and the residual basis (C9). Sequentially. the 1st basis (C1) 11 is produced first, then the 2nd basis (C2) 12, and so on. For data processing, the first eight bases 11˜18 represent the original resting ECG 10. In an embodiment, the first six bases 11˜16 represent the original resting ECG 10.

In an embodiment, an energy index of the basis is |Ci|=√(∫(Ci·Ci)dt). The range of integration is 4 seconds after the monitored signal is stable, or as long as 10 seconds. And the energy index of an resting ECG is Ctotal=√(Σ|Ci|·|Ci|).

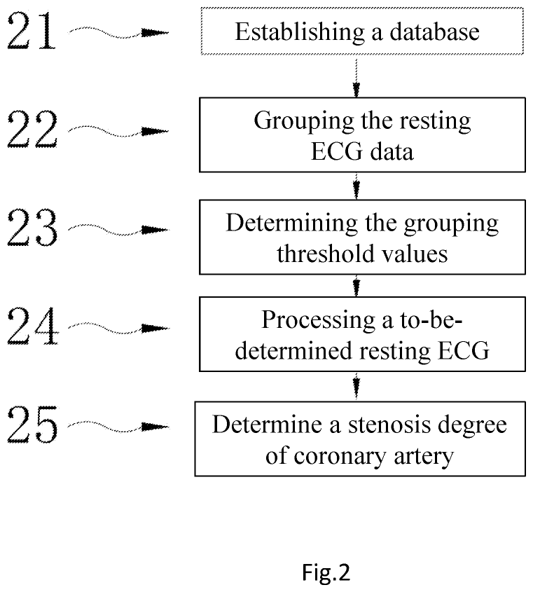

Referred to FIG. 2, in an embodiment, a method for analyzing resting ECG is provided for determining whether there is stenosis of the coronary artery, and the degree of the stenosis The method comprises step 21 to establish the database, step 22 to group in the database, step 23 to determine the threshold value for grouping, step 24 to process the to-be-determined resting ECG, and step 25 to determine the stenosis degree of the coronary artery.

In step 21 and 22, resting ECG data are collected to establish a database and grouped. Some people do the treadmill exercise tests but without doing computer tomography (CT) imaging further, because the cardiologist observe no symptoms and determine no need to do the CT imaging, the CT imaging cost too much for them, or they have no time to do the CT imaging. Their resting ECG data are collected as the reference group, presumed they have no or very slight coronary artery stenosis. They are in the status without the need to have any special medical treatment.

Regarding to the resting ECG accompanying the results of a CT imaging, the degree value of the coronary artery stenosis is determined. If the degree value is above 50%, it is urgent and needs to have the invasive treatment, such as Coronary Stenting. The kind of resting ECGs are collected as the first priority group. If the degree value is below 50%, it may only need medication treatment. Such ECGs are collected as the second priority group. Therefore, according to the degree of coronary artery stenosis, there are three groups in the database, the reference group with no need of special medical treatment, the first priority group with the need of the urgent treatment, and the second group without the need of urgent treatment.

In an embodiment, in the database, the resting ECGs without accompanying CT imaging are collected as the reference group, with no need of special medical treatment. The resting ECGs accompanying CT imaging are collected as the priority group which may need special medical treatment.

In an embodiment, EMD is adopted to decompose any resting ECG in the database into the 1^{st} to 8^{th} basis, and their corresponding energy indexes, |C1| to |C8|, are calculated, as well as the Ctotal. Therefore, every resting ECG data in the database comprises at least the corresponding energy index besides the original resting ECG. In addition, every resting ECG data in the first and second priority groups further comprises the degree value of the coronary artery stenosis.

In an embodiment, every resting ECG data in the database comprises at least the corresponding energy index besides the original resting ECG. In addition, every resting ECG data in the priority group further comprises the degree value of the coronary artery stenosis.

In step 23, the threshold values for grouping are determined. Every resting ECG data needs an corresponding active index to be calculated by (W1·|C1|+W2·|C2|+W3·|C3|)/|Ctotal|, wherein W1, W2, and W3 are the weighting parameters of |C1|, |C2|, and |C3| respectively. Regarding to each group, the active indexes of the collected resting ECG data are summed up and then averaged to obtain the grouping threshold value VM1 for the reference group, VM2 for the first priority group, and VM3 for the second priority group.

In an embodiment, the grouping threshold value of the reference group is VM1, and the grouping threshold value of the priority group is VM2.

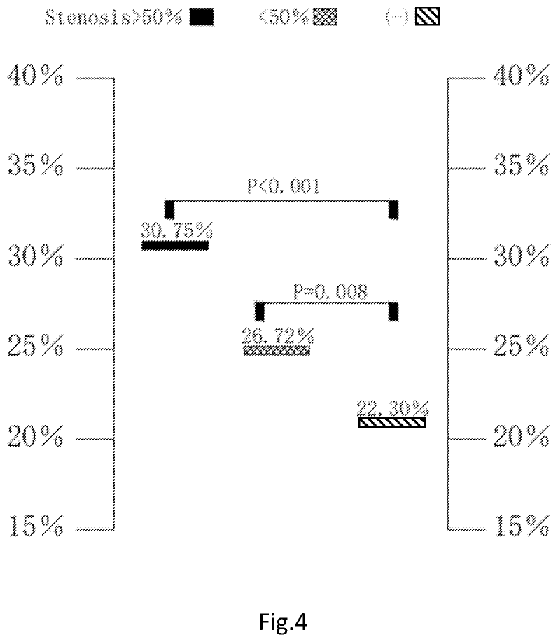

The weighting parameters W1, W2, and W3, are determined according to the following rules: (1) (W1+W2+W3) is less than 1. (2) W1≥W2≥W3. (3) There is a statistically significant difference between VM2 and VM1, as well as VM3 and VM1. In another word, the statistical correlation between VM2 and VM1, as well as VM3 and VM1, is less than 0.05. In an embodiment, W1 is 0.3˜0.4, W2 is 0.3˜0.4, W3 is 0.1˜0.2. Referred to FIG. 4, VM1 is 22.30%, VM2 is 30.75%, VM3 is 26.72%. The statistical correlation between VM3 and VM1 is less than 0.001, and the statistical correlation between VM2 and VM1 is less than 0.008.

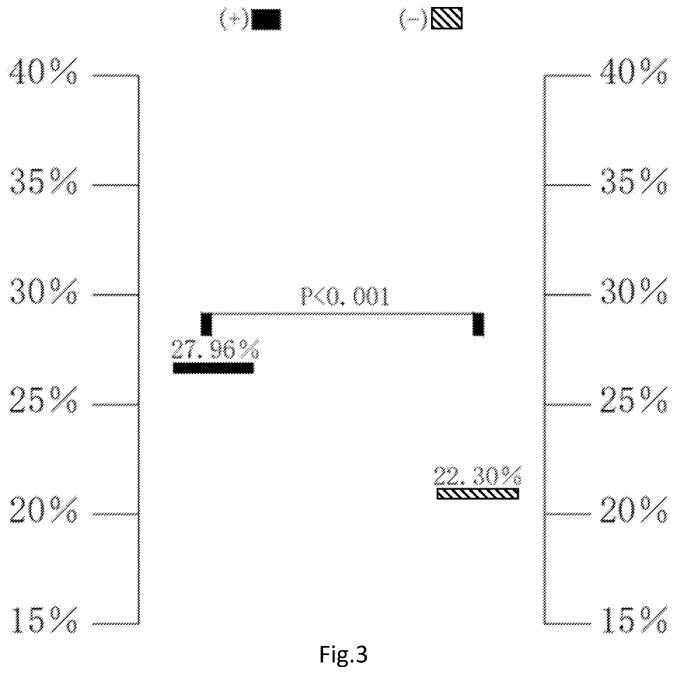

Referred to FIG. 3, in an embodiment, the grouping threshold value of the reference group VM1 is 22.30%, and the grouping threshold value of the priority group VM2 is 27.96%. The statistical correlation between VM2 and VM1 is less than 0.001.

After determining the grouping threshold values and weighting parameters, every resting ECG data with the degree value of the coronary artery stenosis further comprises the corresponding active index in the database. Therefore, the corresponding relation between the active indexes and the degree values of the coronary artery stenosis can be established.

In step 24, a to-be-determined resting ECG is processed. In an embodiment, EMD is adopted to decompose an external to-be-determined resting ECG into eight bases to calculate the corresponding to-be-determined active index.

In step 25, the degree of coronary artery stenosis is determined. The to-be-determined active index is compared with the grouping threshold values. In an embodiment, if the active index is lower than 22.30%, it is in the status without the need of any special medical treatment. If the active index is between 22.30% and 26.72%, it is in the status without the need of the urgent medical treatment. If the active index is above 26.72%, it is in the status with the need of the urgent medical treatment.

In an embodiment, if the active index is lower than 22.30%, it is in a status without the need of special medical treatment. Otherwise, it is in a status with the need of special medical treatment.

In an embodiment, it is available to determine the degree value of the coronary artery stenosis by the to-be-determined active index, according to the established relation between the active indexes and the degree values of the coronary artery stenosis.

What is claimed is:

1. A method for analyzing myocardial ischemia in resting electrocardiogram (ECG), comprising:
establishing a database, comprising to collect a plurality of resting ECG data, wherein each of the resting ECG data comprises a resting ECG and decomposed into a plurality of Intrinsic Mode Functions (IMFs) as a plurality of corresponding bases C1, C2 . . . , and Cn, wherein |Ci|=√(∫Ci·Ci)dt), i=1, 2, . . . , n, n is a nature number, and Ctotal=√(Σ|Ci|·|Ci|), wherein the corresponding bases at least comprises C1, C2, and C3;
grouping the resting ECG data, wherein a reference group is formed by the resting ECGs without any result of any computer tomography (CT) imaging, wherein a first priority group is formed by the resting ECGs accompanying with a plurality of first results of corresponding CT imaging, and each of the first results comprises a corresponding degree value of a first coronary artery stenosis above an stenosis threshold value, wherein a second priority group is formed by the resting ECGs accompanying with a plurality of second results of corresponding CT imaging, and each of the second results comprises a corresponding degree value of a second coronary artery stenosis below an stenosis threshold value;
calculating a corresponding active index of each of the resting ECG data in the database, defined as (W1·|C1|+W2·|C2|+W3·|C3|)/|Ctotal|, wherein W1, W2, W3 are the weighting parameters of C1, C2, C3 respectively, then each active index are summed up to be averaged regarding to the reference group, the first priority group, and the second priority group respectively to obtain a reference grouping threshold value VM1, a first priority grouping threshold value VM2, and a second priority grouping threshold value VM3 respectively, wherein the W1, W2, and W3 are determined according a plurality of rules: (1) (W1+W2+W3)≤1, (2) W1≥W2≥W3, (3) there is a statistically significant difference between VM2 and VM1, and between VM3 and VM1;
receiving a to-be-determined resting ECG, and calculating a corresponding to-be-determined active index; and comparing the corresponding to-be-determined active index with the VM1, VM2, and VM3 to determine a stenosis degree of coronary artery.

2. The method for analyzing resting ECG as claim 1, wherein the stenosis threshold value is 50%.

3. The method for analyzing resting ECG as claim 1, wherein EMD is adopted to produce the IMFs.

4. The method for analyzing resting ECG as claim 1, wherein the corresponding degree value of the first coronary artery stenosis and the corresponding degree value of the second coronary artery stenosis are represented in a percentage form.

5. The method for analyzing resting ECG as claim 4, wherein an active-index-to-degree-value-of-coronary-artery-stenosis relation is established regarding to the first priority group and the second priority group respectively.

6. A method for analyzing myocardial ischemia in resting electrocardiogram (ECG), comprising:
establishing a database, comprising to collect a plurality of resting ECG data, wherein each of the resting ECG data comprises a resting ECG and decomposed into a plurality of Intrinsic Mode Functions (IMFs) as a plurality of corresponding bases C1, C2 . . . , and Cn, wherein |Ci|=√(∫(Ci·Ci)dt), i=1, 2, . . . , n, n is a nature number, and Ctotal=√(Σ|Ci|·|Ci|), wherein the corresponding bases at least comprises C1, C2, and C3;
grouping the resting ECG data, wherein a reference group is formed by the resting ECGs without any result of any computer tomography (CT) imaging, wherein a priority group is formed by the resting ECGs accompanying with a plurality of results of corresponding CT imaging, and each of the results comprises a corresponding degree value of a coronary artery stenosis;
calculating a corresponding active index of each of the resting ECG data in the database, defined as (W1·|C1|+W2·|C2|+W3·|C3|)/Ctotal|, wherein W1, W2, W3 are the weighting parameters of C1, C2, C3 respectively, then each active index are summed up to be averaged regarding to the reference group, the first priority group, and the second priority group respectively to obtain a reference grouping threshold value VM1, a first priority grouping threshold value VM2, and a second priority grouping threshold value VM3 respectively, wherein the W1, W2, and W3 are determined according a plurality of rules: (1) (W1+W2+W3)≤1, (2) W1≥W2≥W3, (3) there is a statistically significant difference between VM2 and VM1, and between VM3 and VM1;
receiving a to-be-determined resting ECG, and calculating a corresponding to-be-determined active index; and comparing the corresponding to-be-determined active index with the VM1, VM2, and VM3 to determine a stenosis degree of coronary artery.

7. The method for analyzing resting ECG as claim 6, wherein EMD is adopted to produce the IMFs.

8. The method for analyzing resting ECG as claim 6, wherein the corresponding degree value of the coronary artery stenosis is represented in a percentage form.
